(19) United States
(12) Patent Application Publication
(10) Pub. No.: US 2008/0026983 A1
(43) Pub. Date: Jan. 31, 2008

(54) Compositions and methods for alleviating joint pain and improving joint flexibility

(76) Inventors: Paul T. Gardiner, Mississauga (CA); Marvin A. Heuer, Mississauga (CA); Shan Chaudhuri, Mississauga (CA); Kenneth Clement, Mississauga (CA)

Correspondence Address: KENYON & KENYON LLP, ONE BROADWAY, NEW YORK, NY, 10004, US

(21) Appl. No.: 11/494,995

(22) Filed: Jul. 28, 2006

(51) Int. Cl. A61K 38/17; A61K 38/46; A61K 31/7048; A61K 31/22; A61K 31/385; A61K 31/12; A61K 31/05

(52) U.S. Cl. 514/2; 514/27; 514/547; 514/440; 514/731; 424/94.65; 514/688

(57) ABSTRACT

A Composition and method for administering a nutritional supplement to an individual, e.g., a human or animal, are provided for the alleviation of joint pain, long-lasting relief from joint pain, improvement of joint functioning as well as help to restore joint flexibility through a reduction of inflammation. The nutritional supplement may include at least Milk Protein Concentrate comprising IgG Hyperimmune micronutrient peptides and source of Curcuminoids. The nutritional supplement may be provided for consumption at least one time daily.

### FIELD OF THE INVENTION

The present invention relates to a nutritional composition for alleviating pain in the joints, restoring joint flexibility and supporting healthy joint function in an individual, e.g., a human or animal.

### SUMMARY OF THE INVENTION

The present invention, according to an embodiment thereof, provides for a nutritional supplement directed towards alleviating joint pain quickly in an effective formulation. The nutritional supplement of the present invention also provides long-lasting relief from joint pain and helps to restore joint flexibility. Said nutritional supplement of the present invention comprises at least Milk Protein Concentrate comprising IgG Hyperimmune micronutrient peptides, e.g., MicroLactin™ and source of Curcuminoids. For example, the present invention may be effective in alleviating joint pain by reducing tight junction permeability as well as inducing a reduction in inflammamation by reducing histamine levels, thus inhibiting vasodilation and permeability of blood vessels at the site of injury.

### DETAILED DESCRIPTION OF THE INVENTION

The present invention according to an embodiment thereof is directed towards a nutritional supplement which may, for example, quickly alleviate joint pain and provide long-lasting relief from joint pain as well as help to restore joint flexibility. The present invention may also be effective in ameliorating the preceding indication by providing a reduction in tight junction permeability leading to a reduction in neutrophil infiltration at the site of injury, a reduction of histamine levels, inhibition of Bradykinin leading to less stimulation of the pain and inflammation pathways, helping to prevent the onset of arthritis, improvement in cell membrane integrity, improvement in joint lubrication, inhibition of pro-inflammatory cytokines as well as providing a source of antioxidants.

In an embodiment, the present invention may include the use of a combination, wherein said combination comprises one or more of, without being limited to, Milk Protein Concentrate comprising IgG Hyperimmune micronutrient peptides, e.g., Microlactin™, a source of Curcuminoids, Bromelain-powder Extract, a source of Epigallocatechin Gallates, Alpha Lipoic Acid, a source of Resveratrol, a source of Esterified Fatty Acid Carbons, a source of casein, and a multimineral joint complex. The supplement dosage may be consumed in any form, e.g., a capsule, a tablet, a caplet, a liquid beverage, a powder beverage mix or as a dietary gel. The preferred dosage of the present embodiment is as a caplet.

Furthermore, the dosage form of the nutritional supplement in accordance with the aforementioned embodiment or further embodiments as interpreted by one of skill in the art related to the present invention may be provided in accordance with customary processing techniques for herbal and/or dietary or nutritional supplements in any of the forms mentioned above. In an embodiment, said nutritional supplement is provided in a timed-release oral mode of delivery.

The present invention may be employed to alleviate joint pain and provide long-lasting relief therefrom while concomitantly improving joint flexibility. The nutritional supplement of the present invention may be of particular interest to those seeking to improve joint function as a result of inflammation as well as relieving the pain associated with joint inflammation. The amount of the composition administered an individual, e.g. a human or an animal, may vary depending upon, e.g., the desired effect, the size of the person, the individual characteristics of the individual, and other such factors. For example, the presented and various other envisioned embodiments of the composition of the present invention may be administered to the diet of a person on a daily basis as needed to ameliorate joint inflammation and the associated pain.

### Milk Protein Concentrate

Milk Protein Concentrate comprising IgG Hyperimmune micronutrient peptides, e.g., MicroLactin™, is a natural ingredient derived from concentrated milk proteins wherein the key constituent provides fast, effective pain relief and improves joint function related to everyday activities (including reduced stiffness and increased flexibility), while promoting joint health (Zenk J L, Helmer T R, Kuskowskl, M A. The Effects of Milk Protein Concentrate on the Symptoms of Osteoarthritis in Adults: An Exploratory, Randomized, Double-Blind, Placebo-Controlled Trial. Curr Ther Res 2002; 63(7); Colker C M, Swain M, Lynch L, Gingerich D A. Effects of a Milk-Based Bioactive Micronutrient Beverage on Pain Symptoms and Activity of Adults with Osteoarthritis: A Double-blind, Placebo-Controlled, Clinical Evaluation. Appl Nutr Inves 2002; 18:388-392; Zenk J L. Prospective, Randomized, Double-Blind Study to Evaluate the Effect of Milk Protein Concentrate for the Relief of Joint Aching, Pain and Stiffness in Subjects with Osteoarthritis: A Two Phase Study (Phase 1 not published, this is a pilot study). The purported benefits of Milk Protein Concentrate comprising IgG Hyperimmune micronutrient peptides, e.g., MicroLactin™, have been observed in as little as 2 weeks (Zenk J L, Helmer T R, Kuskowskl, M A. The Effects of Milk Protein Concentrate on the Symptoms of Osteoarthritis in Adults: An Exploratory, Randomized, Double-Blind, Placebo-Controlled Trial. Curr Ther Res 2002; 63(7); Colker C M, Swain M, Lynch L, Gingerich D A. Effects of a Milk-Based Bioactive Micronutrient Beverage on Pain Symptoms and Activity of Adults with Osteoarthritis: A Double-blind, Placebo-Controlled, Clinical Evaluation. Appl Nutr Inves 2002; 18:388-392), this being significantly earlier than that observed with glucosamine supplementation alone (Zenk J L, Helmer T R, Kuskowskl, M A. The Effects of Milk Protein Concentrate on the Symptoms of Osteoarthritis in Adults: An Exploratory, Randomized, Double-Blind, Placebo-Controlled Trial. Curr Ther Res 2002; 63(7)).

In a clinical study conducted on patients with osteoarthritis (Zenk J L, Helmer T R, Kuskowskl, M A. The Effects of Milk Protein Concentrate on the Symptoms of Osteoarthritis in Adults: An Exploratory, Randomized, Double-Blind, Placebo-Controlled Trial. Curr Ther Res 2002; 63(7)), total pain was improved, e.g., reduced, by over 30% and physical function was improved by over 25% within 6 weeks. Furthermore, joint stiffness was improved by over 40% within 4 weeks (Zenk J L, Helmer T R, Kuskowskl, M A. The Effects of Milk Protein Concentrate on the Symptoms of Osteoarthritis in Adults: An Exploratory, Randomized, Double-Blind, Placebo-Controlled Trial. Curr Ther Res 2002; 63(7)). As such, Milk Protein Concentrate comprising IgG Hyperimmune micronutrient peptides. e.g., MicroLactin™ supplementation was found to be significantly more effective than glucosamine alone over a 6 week period in alleviating joint pain and ameliorating joint physical functioning and stiffness.

Another clinical study, also conducted on patients with osteoarthritis (Colker C M, Swain M, Lynch L, Gingerich D A. Effects of a Milk-Based Bioactive Micronutrient Beverage on Pain Symptoms and Activity of Adults with Osteoarthritis: A Double-blind, Placebo-Controlled, Clinical Evaluation. Appl Nutr Inves 2002; 18:388-392.) showed that the ability to perform daily activities was improved 3.5-fold within 3 weeks following the commencement of supplementation with 5-fold improvements in pain and stiffness seen by the end of the first month based on WOMAC score. The WOMAC Criteria consist of the following: walking stairs, getting up from a seated position, standing, bending to the floor, walking, getting in and out of a car, shopping, putting on or taking off socks, turning over in bed, sitting, getting on and off the toilet, heavy and light domestic duties, general work activities, sitting cross-legged, cycling, driving and praying.

Other benefits of Milk Protein Concentrate comprising IgG Hyperimmune micronutrient peptides, e.g., MicroLactin™, may further comprise cholesterol and blood-pressure lowering (Sharpe S J, Gamble G D, Sharpe D N. Cholesterol-lowering and blood pressure effects of immune milk. Am J Clin Nutr. 1994; 59:929-934). Moreover, a clinical study involving highly trained runners reported that Milk Protein Concentrate comprising IgG Hyperimmune micronutrient peptides, e.g., Microlactin™, supplementation resulted in significantly reduced muscle recovery times following strenuous activity as compared to control milk (Krick G H, M D, Cedar Medical Center; Wilson R, Professor of Exercise Physiology, University of Puget Sound; Wilbur K, Study Coordinator, Tacoma, Wash., U.S.A. Double-blind Controlled Trial on the Effect of Stolle Milk on Recovery after Exercise in Highly Trained Runners).

Milk Protein Concentrate comprising IgG Hyperimmune micronutrient peptides, e.g., MicroLactin™, is thought to act by decreasing inflammation in joints via a reduction in tight junction permeability (Ormord D. J., Miller T. E. A low molecular weight component derived from the milk of yper immunized cows suppresses inflammation by inhibiting neutrophil emigration. Agents & Actions 1992; 35:1-10). This, in turn, inhibits neutrophil migration from blood vessels into the vascular space within joints (Stelwagen K., Ormord D. J. An anti-inflammatory component derived from milk of hyperimmunized cows reduces tight junction permeability in vitro. Inflammation Research 1998; 47:384-388), which causes inflammation resulting in reduced joint mobility, stiffness and pain. Moreover, Milk Protein Concentrate comprising IgG Hyperimmune micronutrient peptides, e.g., MicroLactin™ acts to restore the protective capillary barrier around joints (Zenk J L, Helmer T R, Kuskowskl, M A. The Effects of Milk Protein Concentrate on the Symptoms of Osteoarthritis in Adults: An Exploratory, Randomized, Double-Blind, Placebo-Controlled Trial. Curr Ther Res 2002; 63(7)).

One aspect of the present invention includes the use of IgG Hyperimmune micronutrient milk anti-inflammatory peptides. For example, U.S. Pat. Nos. 4,284,623, 4,956,349, 5,194,255, 5,242,691, 5,352,462, 5,650,175, and 5,980,953 disclose methods and processes related to the extraction and use of said IgG Hyperimmune micronutrient milk anti-inflammatory peptides.

U.S. Pat. No. 4,284,623 purports to describe a process for producing an anti-inflammatory milk and further discloses a method for treating inflammation in animals. The patent purports to describe a process of immunizing a bovid involving the steps of antigen selection, sensitization of the bovid by primary immunization, testing the serum of the bovid to confirm sensitivity induction, administering boosters of the appropriate dosage to induce and maintain an anti-inflammatory factor-producing state, testing the properties of the milk and collecting milk from the bovid during the anti-inflammatory factor-producing state.

U.S. Pat. No. 4,956,349 purports to describe a method of producing an anti-inflammatory factor from bovid milk by maintaining the animal in a hyperimmunized state using a polyvalent mixture of bacterial antigens. Further, the patent suggests a method of treating inflammation in an animal which comprises administering to the animal an anti-inflammatorily effective amount of the anti-inflammatory factor termed “Milk Anti-inflammatory Factor (MAIF)”.

U.S. Pat. No. 5,194,255 purport to describe a method for reducing elevated arterial blood pressure in an animal utilizing an anti-hypertensive factor from hyperimmunized milk. The bovid from which the Hyperimmune milk is obtained is immunized using a heat-killed polyvalent bacterial liquid vaccine.

U.S. Pat. No. 5,242,691 purports to describe a method and use of the isolation of an anti-inflammatory composition from hyperimmunized milk. The anti-inflammatory composition is produced by a process involving the following steps of: removing the fat from milk of a milk-producing animal to produce skimmed milk, pasteurizing the skimmed milk, removing casein from the resultant milk, removing the whey macromolecules with a weight greater than 10,000 Daltons, reducing the ionic strength of the composition to produce an aggregate with anti-inflammatory activity wherein the aggregate has a molecular weight of less than 5,000 Daltons, removing from the composition the macromolecules having a weight less than about 5,000 Daltons, and collecting the composition. The patent also purports to describe a method of inhibiting a cellular inflammatory response by administering to an animal the anti-inflammatory composition.

U.S. Pat. No. 5,352,462 purports to describe a method for using anti-inflammatory milk to prevent interaction between CD18 cell-surface antigens and other molecules where these interactions are known to be necessary for cells to exit the vasculature and that such an emigration leads to increased tissue damage in animals during the inflammatory response. The invention also claims the use of the anti-inflammatory factor in mammals to prevent the emigration of cells from the vasculature and to suppress the mitogenic response of lymphocytes to foreign antigens. This patent also purports to describe a method for detaching neutrophils which have attached to endothelial cells in a mammal using the anti-inflammatory factor.

U.S. Pat. No. 5,650,175 purports to describe a use of anti-inflammatory factor present in milk purified by filtering to remove molecules with weight greater than 10,000 Daltons, fractionating the filtrate by ion-exchange, gel filtration and affinity chromatography. The purported invention is also directed towards a method for using milk anti-inflammatory factor to prevent neutrophils from adhering to the endothelium of venules or to detach neutrophils which have already adhered to the endothelial cells lining the walls of the venules, thereby reducing the tissue damage associated with the inflammatory response.

U.S. Pat. No. 5,980,953 purports to describe additional methods for purifying an anti-inflammatory factor from skimmed milk involving the following steps of: ultra-filtering skimmed milk through a filter with a molecular weight cut-off of 1,000 Daltons, collecting the less than 1,000 Dalton permeate, extracting the permeate by organic partition extraction and obtaining the organic extract from the extraction, separating the organic extract by reverse-phase HPLC chromatography and collecting the eluate. The patent also purports to describe methods for inhibiting neutrophil adherence and detaching neutrophils from endothelial walls, inhibiting CD18 interactions, inhibiting the emigration of leukocytes, inhibiting an inflammatory response, and suppressing the mitogenic response of lymphocytes in a host mammal to foreign antigens.

In an embodiment of the present invention, which is set forth in greater detail in Example 1 below, the nutritional supplement may include Milk Protein Concentrate comprising IgG Hyperimmune micronutrient peptides. A serving of the nutritional supplement may include from about 1 mg to about 10 g of Milk Protein Concentrate comprising IgG Hyperimmune micronutrient peptides. The preferred dosage of a serving of the nutritional supplement comprises about 2.000 g of Milk Protein Concentrate comprising IgG Hyperimmune micronutrient peptides.

### Curcuminoids

Curcumin is the active compound of tumeric (Curcumin longa), which is a key ingredient in many curries.

Curcumin, when used to treat post-surgical inflammation, has been shown to be superior to some traditional medications (Satoskar R R, Shah S J, Shenoy S G. Evaluation of anti-inflammatory property of curcumin (diferuloyl methane) in patients with postoperative inflammation. International Journal of Clinical Pharmacology, Therapy, and Toxicology, 1986; 24, 651-654). Additionally, in mice, it has been shown to offer a potential treatment with respect to neuropathic pain by acting through the inhibition of nitric oxide (NO) and tumor necrosis factor-alpha (TNFα) (Sameer Sharma, Srinivas K. Kulkarni, Javed N. Agrewala and Kanwaljit Chopra. Curcumin attenuates thermal hyperalgesia in a diabetic mouse model of neuropathic pain. Eur J Pharmacol. 2006 May 1; 536(3):256-61. Epub 2006 March 13). Curcuminoids have furthermore been shown to protect against free radical damage via their action as a potent antioxidants (Sreejayan N, Rao M N A. Free radical scavenging activity of curcuminoids. Arzneimittelforschung, 1996; 46, 169-171.) and they are also able to reduce inflammation through the reduction of circulating histamine levels (Arora R B, Basu N, Kapoor V, Jain A P. Anti-inflammatory studies on Curcuma longa (turmeric). Indian Journal of Medical Research, 1971; 59, 1289-1295).

In an embodiment of the present invention, which is set forth in greater detail in Example 1 below, the nutritional supplement may include Curcumin. A serving of the nutritional supplement may include from about 1 mg to about 1 g of Curcumin. The preferred dosage of a serving of the nutritional supplement comprises about 0.005 g of Curcumin.

### Bromelain

Bromelain is a proteolytic enzyme found in pineapple which has been shown to be an effective anti-inflammatory agent in rats, even when compared to prednisone (Uhlig G, Seifert J. The effect of proteolytic enzymes (traumanase) on posttraumatic edema. Fortschr Med 1981; 99:554-556, Vellini M, Desideri D, Milanese A, et al. Possible involvement of eicosanoids in the pharmacological action of bromelain. Arzneimittelforschung 1986; 36:110-112), a conventional steroidal anti-inflammatory. It is often used as a digestive aid as well as to reduce tissue and muscular inflammation. The action of Bromelain has been evidenced to be due to its inhibitory action on Bradykinin, a stimulator of pain and inflammation (Kumakura S, Yamashita M, Tsurufuji S. Effect of bromelain on kaolin-induced inflammation in rats. Eur J Pharmacol 1988; 150:295-301). In rats, Bromelain also has been shown to stimulate the conversion of plasminogen to plasmin which in turn increases venous permeability and thus aiding in drainage, thereby facilitating anti-inflammatory action (Taussig S J, Batkin S. Bromelain, the enzyme complex of pineapple (Ananas comosus) and its clinical application. An update. J Ethnopharmacol 1988; 22:191-203). An early clinical trial demonstrated that Bromelain supplementation results in a drastic reduction in swelling in boxers as compared to placebo (Blonstein J L. Control of swelling in boxing injuries. Practitioner 1960; 185:78). In another early study, supplementation resulted in greatly improved post-surgical recovery in terms of pain and swelling (Tassman G C, Zafran J N, Zayon G M. Evaluation of a plant proteolytic enzyme for the control of inflammation and pain. J Dent Med 1964; 19:73-77). Bromelain has been shown to benefit the recovery of blunt musculoskeletal injuries in all 4 parameters of: swelling, pain at rest and during movement, and tenderness, examined in an additional study (Masson M. Bromelain in blunt injuries of the locomotor system. A study of observed applications in general practice. Fortschr Med 1995; 113:303-306).

In an embodiment of the present invention, which is set forth in greater detail in Example 1 below, the nutritional supplement may include Bromelain-powder Extract. A serving of the nutritional supplement may include from about 1 mg to about 1 g of Bromelain-powder Extract. The preferred dosage of a serving of the nutritional supplement comprises about 0.001 g of Bromelain-powder Extract.

### Green Tea Leaf

Epigallocatechin Gallate (EGCG) is the most active Catechin in Green Tea (Camellia sinensis) leaf and is mainly used as a weight loss agent.

Green tea, has been shown to reduce total cholesterol levels (Kono S, Shinchi K, Ikeda N. Green tea consumption and serum ipid profiles: A cross-sectional study in Northern Kyushu, Japan. Preventive Medicine, 1992; 21, 526-531.) and to promote fat reduction as well as increase energy expenditure in humans (Dulloo, A. G. Efficacy of a green tea extract rich in catechin polyphenols and caffeine in increasing 24-h energy expenditure and fat oxidation in humans. American Journal of Clinical Nutrition, 1999; 70, 1040-1045; Chantre P, Lairon D. Recent findings of green tea extract R25 (Exolise) and its activity for the treatment of obesity. Phytomedicine, 2002; 9, 3-8.) by way of both blocking fat absorption and boosting metabolic rate. Furthermore, in mice, the EGCG-containing fraction of green tea was found to prevent the onset of and furthermore, showed a reduction in the severity of arthritis (Haqqi T M, Anthony D D, Gupta S, et al. Prevention of collagen-induced arthritis in mice by a polyphenolic fraction from green tea. Proc Natl Acad Sci U S A, 1999 Apr. 13; 96(8):4524-9).

In an embodiment of the present invention, which is set forth in greater detail in Example 1 below, the nutritional supplement may include Green Dry Leaf Extract. A serving of the nutritional supplement may include from about 1 mg to about 1 g of Green Dry Leaf Extract. The preferred dosage of a serving of the nutritional supplement comprises about 0.001 g of Green Dry Leaf Extract.

### Alpha Lipoic Acid (ALA)

Alpha Lipoic Acid, also commonly known as 6,8-thioctic acid is both a water- and fat-soluble enzyme found in mitochondria. It possesses strong antioxidant activity.

ALA supplementation has been shown to increase glucose clearance in patients with type-2 diabetes (Jacob S, Henriksen E J, Schiemann A L, Simon I, Clancy D E, Tritschier H J, Jung W I, Augustin H J, Dietze G J. Enhancement of glucose disposal in patients with type 2 diabetes by alpha-lipoic acid. Arzneimittelforschung, 1995; 45, 872-87; Jacob S, Ruus P, Hermann R, Tritschler H J, Maerker E, Renn W, Augustin H J, Dietze G J, Rett K. Oral administration of RAC-alpha-lipoic acid modulates insulin sensitivity in patients with type-2 diabetes mellitus: a placebo-controlled pilot trial. Free Radical Biology and Medicine, 1999; 27, 309-314). Moreover, its antioxidant activities (Packer L, Witt E H, and Tritschler H J. Alpha-Lipoic acid as a biological antioxidant. Free Rad Biol Med 1995; 19: 227-250.) have been shown to improve glucose tolerance and insulin-sensitivity in insulin-resistant obese Zucker rats (Saengsirisuwan V, Kinnick T R, Schmit M B, and Henriksen E J. Interactions of exercise training and -lipoic acid on glucose transport in obese Zucker rats. J Appl Physiol 2001; 91: 145-153). This increase in insulin-sensitivity has been shown to be further improved with exercise and may be associated with an increase in the insulin receptor substrate 1 (IRS1) as well as its association with the p85 regulatory subunit of phosphatidylinositol 3-kinase (Saengsirisuwan V. Perez F R, Sloniger J A, Maier T, and Henriksen E J. Interactions of exercise training and -lipoic acid on insulin signaling in skeletal muscle of obese Zucker rats. Am J Physiol Endocrinol Metab 2004; 287: E529-E536).

In an embodiment of the present invention, which is set forth in greater detail in Example 1 below, the nutritional supplement may include Alpha Lipoic Acid. A serving of the nutritional supplement may include from about 1 mg to about 1 g of Alpha Lipoic Acid. The preferred dosage of a serving of the nutritional supplement comprises about 0.001 g of Alpha Lipoic Acid.

### Polygonum cuspidatum

Resveratrol, found in Polygonum cuspidatum, is a natural phytoalexin produced by some plants as response to injury or fungal infections. It is found in high concentrations in grape skins and is used to prevent heart disease.

Many clinical studies have been undertaken to examine the anti-cancer and heart benefits of Resveratrol. Most of the reported benefits stem from in vitro work in addition to and the “French paradox” wherein the observation was made that France's population, despite a diet high in fat, present with a low incidence of heart disease (Burkitt M J, Duncan J. Effects of trans-resveratrol on copper-dependent hydroxyl-radical formation and DNA damage: Evidence for hydroxyl-radical scavenging and a novel. Glutathione-sparing mechanism of action. Arch Biochem Biophys. 2000; 381:253-263). Resveratrol has also received attention due to its display of anti-cancer properties (Jang M, Cai L, Udeani G O, Slowing K V, Thomas C F, Beecher C W, Fong H H, Farnsworth N R, Kinghorn A D, Mehta R G, Moon R C, Pezzuto J M. Cancer chemopreventive activity of resveratrol, a natural product derived from grapes. Science, 1997; 275:218-220.) which may be possibly acting through its action as a potent antioxidant (Kopp P. Resveratrol, a phytoestrogen found in red wine. A possible explanation for the conundrum of the ‘French paradox’? European Journal of Endocrinology 1998; 138:619-620; Chanvitayapongs S, Draczynska-Lusiak B, Sun A Y. Amelioration of oxidative stress by antioxidants and resveratrol in PC12 cells. Neuroreport, 1997; 8:1499-1502; Belguendouz L, Fremont L, Gozzelino M T. Interaction of transresveratrol with plasma lipoproteins. Biochemical Pharmacology, 1998; 55:811-816; Frankel E N, Waterhouse A L, Kinsella J E. Inhibition of human LDL oxidation by resveratrol. Lancet, 1993; 341:1103-1104).

There are a number of mechanisms which may account for the beneficial effects of Resveratrol. The ability of Resveratrol to inhibit low density lipoprotein (LDL) oxidation (Frémont L, Belguendouz L, Delpal S. Antioxidant activity of resveratrol and alcohol-free wine polyphenols related to LDL oxidation and polyunsaturated fatty acids. Life Sci. 1999; 64:2511-2521.) may be its primary mechanism of action in preventing heart disease. Resveratrol has additionally been shown to inhibit smooth muscle cell proliferation (Zou J, Huang Y, Chen Q, et al. Suppression of mitogenesis and regulation of cell cycle traverse by resveratrol in cultured smooth muscle cells. Int J Oncol. 1999; 15:647-651; Poussier B, Cordova A C, Becquemin J P, Sumpio B E. Resveratrol inhibits vascular smooth muscle cell proliferation and induces apoptosis. J Vasc Surg. 2005 December; 42(6):1190-7), which is a factor considered necessary for arterial plaque formation again, possibly acting towards the prevention of heart disease. Resveratrol has further been shown to induce CYP1A1 enzyme activity, which is known to protect against disease and oxidative stress (Ciolino H P, Yeh G C. Inhibition of aryl hydrocarbon-induced cytochrome P450 1A1 enzyme activity and CYP1A1 expression by resveratrol. Mol Pharmacol. 1999; 56:760-767).

In an embodiment of the present invention, which is set forth in greater detail in Example 1 below, the nutritional supplement may include Polygonum Cuspidatum-Powder Extract. A serving of the nutritional supplement may include from about 1 mg to about 1 g of Polygonum Cuspidatum-Powder Extract. The preferred dosage of a serving of the nutritional supplement comprises about 0.001 g of Polygonum Cuspidatum-Powder Extract.

Esterified Fatty Acid Carbons (e.g. Celadrin)

Esterified fatty acid carbons are naturally occurring fatty acids used to promote cardiovascular and joint health. In a clinical study, utilizing patients with osteoarthritis of the knee, a significant improvement in knee flexion was demonstrated with Celadrin supplementation versus a placebo control (Hesslink R Jr, Armstrong D 3rd, Nagendran M V, Sreevatsan S, Barathur R. Cetylated fatty acids improve knee function in patients with osteoarthritis. J Rheumatology 2002; 8:1708-1712). Another clinical study, using Esterified Fatty Acid Carbons e.g., Celadrin, as a topical cream, found a significant increase in physical performance associated with treatment versus a placebo in as little as 30 minutes (Kraemer W J, Ratamess N A, Anderson J M, Maresh C M, Tiberio D P, Joyce M E, Messinger B N, French D N, Rubin M R, Gomez A L, Volek J S, Hesslink R Jr. Effect of a cetylated fatty acid topical cream on functional mobility and quality of life of patients with osteoarthritis. J Rheumatology 2004; 4:767-74). A follow-up study found that combining Esterified Fatty Acid Carbons e.g., Celadrin, with menthol (again, as a topical cream) displayed similar improvements in both increased physical activity and reduction in pain (Kraemer W J, Ratamess N A, Maresh C M, Anderson J A, Volek J S, Tiberio D P, Joyce M E, Messinger B N, French D N, Sharman M J, Rubin M R, Gomez A L, Silvestre R, Hesslink R L Jr. A cetylated fatty acid topical cream with menthol reduces pain and improves functional performance in individuals with arthritis. J Strength Cond Res. 2005 May; 19(2):475-80).

The mechanisms of action of the fatty acids which comprise Esterified Fatty Acid Carbons e.g., Celadrin are thought to improve the overall integrity of cell membranes as well as aid in the lubrication of joints.

In an embodiment of the present invention, which is set forth in greater detail in Example 1 below, the nutritional supplement may include Esterified Fatty Acid Carbons, e.g., Celadrin. A serving of the nutritional supplement may include from about 1 mg to about 1 g of Esterified Fatty Acid Carbons e.g., Celadrin. The preferred dosage of a serving of the nutritional supplement comprises about 0.001 g of Esterified Fatty Acid Carbons, e.g., Celadrin.

### Casein (C-12 Peptide)

The C12 Casein-derived natural milk-derived protein is used in methods of controlling blood pressure.

Clinical studies indicate that C-12 may be effective at controlling hypertension by reducing both systolic and diastolic blood pressure (Townsend R R, McFadden C B, Ford V, Cadee J A. A randomized, double-blind, placebo-controlled trial of casein protein hydrolysate (C12 peptide) in human essential hypertension. Am J Hypertens. 2004; November; 17(11 Pt 1):1056-8). The mode of action is thought to be by inhibition of the release of angiotensin II, thereby preventing constriction of blood vessels (Sekiya et al. Journal Jpn. Soc. Nutr. Food Sci., 45, 513-517, (1992-18 participants), Unpublished Univ. of Penn. Study (2002, 10 participants).) which leads to hypertension.

In an embodiment of the present invention, which is set forth in greater detail in Example 1 below, the nutritional supplement may include Casein (C-12 Peptide). A serving of the nutritional supplement may include from about 1 mg to about 1 g of Casein (C-12 Peptide). The preferred dosage of a serving of the nutritional supplement comprises about 0.001 g Casein (C-12 Peptide).

### Multimineral Joint Complex

In a recent randomized control trial, it was suggested that supplementation with a mulitmineral joint complex can improve joint health and function within 1-2 weeks (Miller M J, Mehta K, Kunte S, Raut V, Gala J, Dhumale R, Shukla A, Tupalli H, Parikh H, Bobrowski P, Chaudhary J. Early relief of osteoarthritis symptoms with a natural mineral supplement and a herbomineral combination: a randomized controlled trial. J Inflamm (Lond). 2005; October 21; 2:11). The mechanism of action is suspected to act via the inhibition of the pro-inflammatory cytokine, IL-1β (Miller M J S, Ahmed S, Bobrowski P, Haqqi T M: Suppression of human cartilage degradation and chondrocyte activation by a unique mineral supplement (sierrasil™) and a cat's claw extract, vincaria®. J Amer Nutr Assoc 2004; 7:32-39.) however experimentally this still remains unknown.

In an embodiment of the present invention, which is set forth in greater detail in Example 1 below, the nutritional supplement may include a naturally-occurring mineral composite, e.g., SierraSil™ joint complex. A serving of the nutritional supplement may include from about 1 mg to about 1 g of a multimineral joint complex. The preferred dosage of a serving of the nutritional supplement comprises about 0.001 g of a naturally-occurring mineral composite, e.g., SierraSil™ joint complex.

The present invention, according to an embodiment thereof, provides a method which includes the step of consuming a composition, wherein the method may lead to quick alleviation of joint pain and provide long-lasting relief from joint pain as well as help to restore joint flexibility via a reduction in inflammation. The present invention may additionally provide via execution of the method sources of antioxidants. According to an embodiment of the present invention as well as others envisioned by those of skill in the art, the method includes the step of consuming a nutritional supplement which reduces inflammation thereby ameliorating joint function as well as providing a source of antioxidants.

In an embodiment of the present invention, the method includes the daily consumption of a combination wherein the combination includes one or more of, without being limited to, IgG Hyperimmune micronutrient peptides i.e., Microlactin™, a source of Curcuminoids, Bromelain-powder Extract, a source of Epigallocatechin Gallates, Alpha Lipoic Acid, a source of Resveratrol, a source of Esterified Fatty Acid Carbons, a sources of casein, and a multimineral joint complex.

The present nutritional supplement or those similarly envisioned by one of skill in the art, may be utilized in methods to quickly alleviate joint pain and provide long-lasting relief from joint pain as well as help to restore joint flexibility via a reduction in inflammation in a formulation designed to be consumed on a daily basis.

Although the following example illustrates the practice of the present invention in one of its embodiments, the example should not be interpreted as limiting the scope of the invention. Other embodiments of the present invention will be apparent to those of skill in the art from consideration of the specification and example.

### EXAMPLE

A dietary supplement is provided in two servings per day as caplets. The formulation comprises Milk Protein Concentrate comprising IgG Hyperimmune micronutrient peptides, e.g., Microlactin™, (2.000 g), Curcumin Longa-Powder Extract (0.005 g) standardized to 95% Curcuminoids, Bromelain-powder Extract (0.001 g), Green Tea Dry Leaf Extract (0.001 g) standardized to 45% EGCG, Alpha Lipoic Acid (0.001 g), Polygonum Cuspidatum-Powder Extract (0.001 g) standardized to 50% Resveratrol, Esterified Fatty Acid Carbons (0.001 g) (Celadrin), Casein as a source of C-12 peptide (0.001 g), and a naturally-occurring mineral composite, e.g., SierraSil™, joint complex (0.001 g).

Directions: As a dietary supplement, 2 caplets are administered with an 8 oz. glass of water twice daily. Preferably, each two caplet serving may be consumed with each meal, e.g., breakfast, lunch and dinner.

What is claimed is:

1. A nutritional supplement comprising at least Milk Protein Concentrate comprising IgG Hyperimmune micronutrient peptides and a source of Curcuminoids.

2. The nutritional supplement of claim 1, further comprising Bromelain powder Extract.

3. The nutritional supplement of claim 2, further comprising Esterified Fatty Acid Carbons.

4. The nutritional supplement of claim 3, further comprising Casein C-12 Peptide.

5. The nutritional supplement of claim 4, further comprising a source of Resveratrol.

6. The nutritional supplement of claim 1, further comprising Alpha Lipoic Acid or salts and esters thereof.

7. The nutritional supplement of claim 2, further comprising Alpha Lipoic Acid or salts and esters thereof.

8. The nutritional supplement of claim 1, further comprising a source of Epigallocatechin Gallate.

9. The nutritional supplement of claim 2, further comprising a source of Epigallocatechin Gallate.

10. A method for alleviating joint pain comprising the step of administering the nutritional supplement of claim 1 to an individual at least once per day.

11. A method for providing long-lasting relief from joint pain comprising the step of administering the nutritional supplement of claim 1 to an individual at least once per day.

12. A method for improving joint function comprising the step of administering the nutritional supplement of claim 1 to an individual at least once per day.

13. A method for improving joint mobility comprising the step of administering the nutritional supplement of claim 1 to an individual at least once per day.

14. A method for inducing a reduction in neutrophil infiltration at a site of injury and reducing histamine levels comprising the step of administering the nutritional supplement of claim 1 to an individual at least once per day.

15. A method for reducing inflammation at a site of injury comprising the step of administering the nutritional supplement of claim 1 to an individual at least once per day.

16. The method of claim 10, wherein the nutritional supplement is administered at least once daily.

17. The nutritional supplement of claim 1, wherein said nutritional supplement is provided as a caplet for oral delivery.

18. The nutritional supplement of claim 1 wherein said nutritional supplement is provided in a timed-release oral mode of delivery.
